(19) United States
(12) Patent Application Publication
(10) Pub. No.: US 2021/0074393 A1
(43) Pub. Date: Mar. 11, 2021

(54) MEDICAL CARE SUPPORT DEVICE, MEDICAL CARE SUPPORT METHOD, AND MEDICAL CARE SUPPORT PROGRAM

(71) Applicant: FUJIFILM Corporation, Tokyo (JP)

(72) Inventors: Haruyasu NAKATSUGAWA, Kanagawa (JP); Takeshi FUKUDA, Kanagawa (JP)

(73) Assignee: FUJIFILM Corporation, Tokyo (JP)

(21) Appl. No.: 17/011,672

(22) Filed: Sep. 3, 2020

(30) Foreign Application Priority Data
Sep. 5, 2019 (JP) 2019-162207

(51) Int. Cl. G16H 10/60; G16H 15/00; G16H 50/70; G06Q 30/00; G06Q 50/26; G16H 50/20; G16H 70/20; G06F 21/62; G06F 16/2457

(52) U.S. Cl. CPC G16H 10/60; G16H 15/00; G16H 50/70; G06Q 30/0185; G06F 16/24578; G16H 50/20; G16H 70/20; G06F 21/6245; G06Q 50/265

(57) ABSTRACT

A medical care support device includes a first display control unit that performs control of displaying, on a display unit, electronic medical record information including medical care information used for medical care for a subject and relative health information absence information or health information presence information representing presence or absence of relative health information on health of a relative of the subject, a reception unit that receives a display instruction performed according to the display of the relative health information presence information for displaying the relative health information on the display unit, and a second display control unit that performs control of displaying the relative health information on the display unit in a case where the reception unit receives the display instruction.

### CROSS-REFERENCE TO RELATED APPLICATIONS

The present application claims priority under 35 U.S.C. § 119 to Japanese Patent Application No., 2019-162207 filed on Sep. 5, 2019. The above application is hereby expressly incorporated by reference, in its entirety, into the present application.

### BACKGROUND

### 1. Technical Field of the Invention

The present disclosure relates to a medical care support device, a medical care support method, and a medical care support program.

### 2. Description of the Related Art

In general, health information of a relative of a subject is referred in performing medical care of the subject. For example, JP2015-201092A discloses a technique of displaying a family history highly relevant to a symptom of a patient based on medical information of the relative of the patient.

### SUMMARY

The efficiency of the medical care for the subject can be improved using the information on the health of the relative of the subject which is a medical care target. On the other hand, the information on the health of the relative includes a lot of personal information. Therefore, there is a problem that personal information is disclosed.

In the technique described in JP2015-201092A, there is a case where the personal information is disclosed even though the disclosure of the information is not desired, which is not sufficient from a viewpoint of personal information protection.

The present disclosure has been made in view of the above circumstances, and a purpose thereof is to provide a medical care support device, a medical care support method, and a medical care support program capable of efficiently performing appropriate medical care support for a subject while protecting personal information.

In order to achieve the above object, a medical care support device according to a first aspect of the present disclosure comprises a first display control unit that performs control of displaying, on a display unit, electronic medical record information including first information used for medical care of a subject and second information representing presence or absence of relative health information on health of a relative of the subject, a reception unit that receives a display instruction performed according to the display of the second information to display the relative health information on the display unit, and a second display control unit that performs control for displaying the relative health information on the display unit in a case where the reception unit receives the display instruction.

In the medical care support device according to a second aspect of the present disclosure, the second display control unit displays the relative health information in a case where the reception unit further receives identification information for identifying a person permitted to browse the relative health information, in the medical care support device according to the first aspect.

In the medical care support device according to a third aspect of the present disclosure, the relative health information includes, for each relative of the subject, at least two or more of relative relationship information representing a relationship with the subject, medical history information, medical treatment information, health diagnosis information, or gene information, and the identification information is in correspondence with information that is permitted to be browsed among a plurality of pieces of information included in the relative health information, in the medical care support device according to the second aspect.

In the medical care support device according to a fourth aspect of the present disclosure, the relative health information includes, for each relative, at least one of information representing a relationship with the subject, medical history information, medical treatment information, health diagnosis information, or gene information, in the medical care support device according to the first aspect.

In the medical care support device according to a fifth aspect of the present disclosure, a proposal unit that proposes at least one of an examination related to a disease of the subject which is a medical care target or a medical care policy based on the relative health information is further provided, in the medical care support device according to the first aspect.

In the medical care support device according to a sixth aspect of the present disclosure, the subject is an animal other than a human and a breed of the animal for which the browsing of the relative health information is recommended is determined in advance, and the first display control unit performs control of further displaying, on the display unit, browsing recommended information representing that the browsing is recommended in a case where the breed of the subject is a breed for which the browsing of the relative health information is recommended, in the medical care support device according to the first aspect.

In the medical care support device according to a seventh aspect of the present disclosure, the breed of the animal for which the browsing of the relative health information is recommended is a breed within a predetermined higher order in ranking of at least one of a breeding number or a sales number, in the medical care support device according to the sixth aspect.

In the medical care support device according to an eighth aspect of the present disclosure, an acquisition unit that acquires the relative health information of the subject based on information for identifying a relative in correspondence with a subject which is a medical care target, from correspondence relationship information stored in an information storage unit that stores a correspondence relationship between a plurality of subjects and relative health information of each of the plurality of subjects is further provided, in the medical care support device according to the first aspect.

In the medical care support device according to a ninth aspect of the present disclosure, the information for identifying the relative is information included in a pedigree paper of the subject which is the medical care target, in the medical care support device according to the eighth aspect.

In the medical care support device according to a tenth aspect of the present disclosure, the first display control unit performs control of further displaying, on the display unit, recommended storage information recommending that the correspondence relationship is stored in the information storage unit in a case where the correspondence relationship is not stored in the information storage unit for the subject which is the medical care target, in the medical care support device according to the eighth aspect.

In order to achieve the above object, a medical care support method according to an eleventh aspect of the present disclosure executed by a computer comprises displaying, on a display unit, electronic medical record information including first information used for medical care of a subject and second information representing presence or absence of relative health information on health of a relative of the subject, receiving a display instruction performed according to the display of the second information to display the relative health information on the display unit, and displaying the relative health information on the display unit in a case where the reception unit receives the display instruction.

In order to achieve the above object, a non-transitory computer-readable storage medium storing a medical care support program according to a twelfth aspect of the present disclosure causes a computer to execute the following processing of displaying, on a display unit, electronic medical record information including first information used for medical care of a subject and second information representing presence or absence of relative health information on health of a relative of the subject, receiving a display instruction performed according to the display of the second information to display the relative health information on the display unit, and displaying the relative health information on the display unit in a case where the reception unit receives the display instruction.

The medical care support device according to the present disclosure comprises a memory that stores a command to be executed by a computer, and a processor configured to execute the stored command. The processor displays, on the display unit, the electronic medical record information including the first information used for the medical care of the subject and the second information representing the presence or absence of the relative health information on the health of the relative of the subject, receives the display instruction performed according to the display of the second information to display the relative health information on the display unit, and displays the relative health information on the display unit in the case where the reception unit receives the display instruction.

According to the present disclosure, it is possible to efficiently perform appropriate medical care support for a subject while personal information is protected.

### BRIEF DESCRIPTION OF THE DRAWINGS

FIG. 1 is a block diagram showing an example of a configuration of a medical care support system according to an embodiment.

FIG. 2 is a block diagram showing an example of a hardware configuration of a medical care support device according to the embodiment.

FIG. 3 is a block diagram showing an example of a configuration of an information storage unit according to the embodiment.

FIG. 4A is a table showing an example of relative relationship information according to the embodiment.

FIG. 4B is a table showing an example of health information according to the embodiment.

FIG. 4C is a diagram showing an example of recommended dog breed information according to the embodiment.

FIG. 4D is a table showing an example of authentication information according to the embodiment.

FIG. 5 is a functional block diagram representing an example of a configuration of the medical care support device according to the embodiment.

FIGS. 6A to 6C is a flowchart showing an example of medical care support processing executed by the medical care support device according to the embodiment.

FIG. 7 is a diagram showing an example of electronic medical record information to which browsing recommended information and recommended registration information to be displayed on a display unit according to the embodiment are added.

FIG. 8 is a flowchart showing an example of relative health information registration processing executed in the medical care support processing according to the embodiment.

FIG. 9 is a diagram showing an example of a relative health information registration screen according to the embodiment.

FIG. 10 is a diagram showing an example of electronic medical record information to which relative health information absence information to be displayed on the display unit according to the embodiment is added.

FIG. 11 is a diagram showing an example of electronic medical record information to which relative health information presence information to be displayed on the display unit according to the embodiment is added.

FIG. 12 is a diagram showing an example of an authentication screen according to the embodiment.

FIG. 13 is a diagram showing an example of a pedigree chart according to the embodiment.

FIG. 14 is a diagram showing an example of detailed relative information according to the embodiment.

FIG. 15 is a flowchart showing an example of proposal processing executed by the medical care support device according to the embodiment.

FIG. 16 is a diagram showing an example of medical care proposal information according to the embodiment.

FIG. 17 is a diagram showing another example of the electronic medical record information to which the browsing recommended information and the recommended registration information to be displayed on the display unit according to the embodiment are added.

### DETAILED DESCRIPTION

Hereinafter, an embodiment for implementing a technique of the present disclosure will be described in detail with reference to drawings. In the following embodiment, a case where “dog” is employed as a subject will be described.

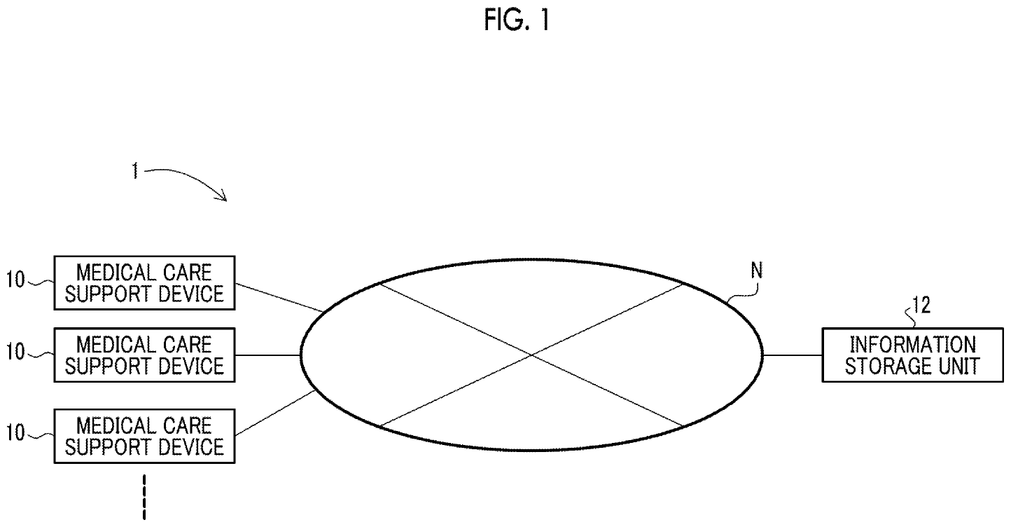

First, a medical care support system 1 according to the present embodiment will be described with reference to FIG. 1. FIG. 1 is a block diagram representing an example of a configuration of the medical care support system 1 according to the present embodiment. As shown in FIG. 1, the medical care support system 1 according to the present embodiment comprises a plurality of (three in FIG. 1 as an example) medical care support devices 10 and an information storage unit 12. The plurality of medical care support devices 10 and the information storage unit 12 are respectively connected to a network N and can communicate with each other through the network N.

The medical care support device 10 is installed in, for example, an animal hospital and used by a user such as a veterinarian. There is a case where a plurality of medical care support devices 10 are disposed in one animal hospital. Examples of the medical care support device 10 include a personal computer and a tablet computer.

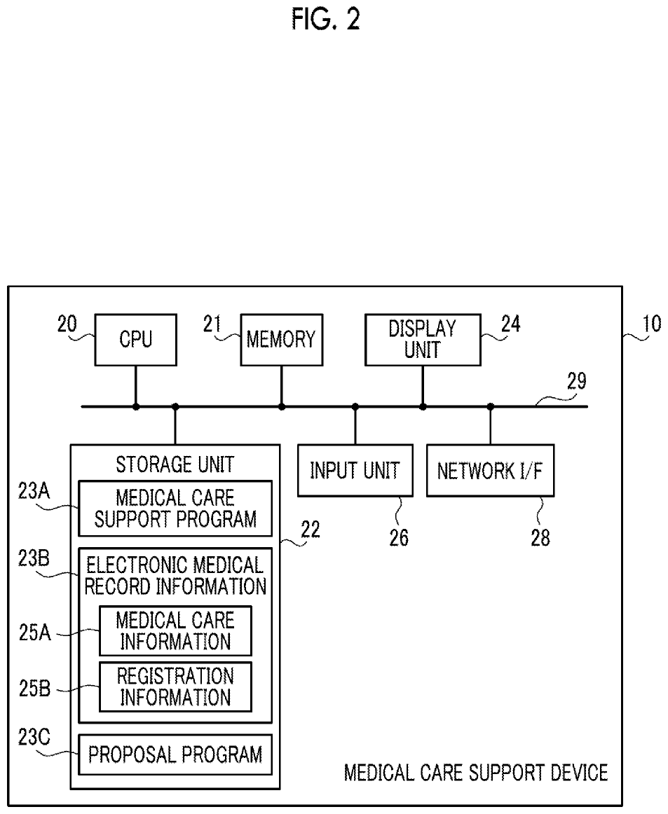

An example of a hardware configuration of the medical care support device 10 according to the present embodiment will be described with reference to FIG. 2. As shown in FIG. 2, the medical care support device 10 includes a central processing unit (CPU) 20, a memory 21 as a temporary storage area, and a nonvolatile storage unit 22. The medical care support device 10 includes a display unit 24 such as a liquid crystal display, an input unit 26 such as a keyboard or a mouse, and a network interface (I/F) 28 connected to the network N. The display unit 24 and the input unit 26 may be integrated as a touch panel display. The CPU 20, the memory 21, the storage unit 22, the display unit 24, the input unit 26, and the network I/F 28 are connected to a bus 29 communicably with each other.

The storage unit 22 is formed by a hard disk drive (HDD), a solid state drive (SSD), a flash memory, and the like. The storage unit 22 as a storage medium stores a medical care support program 23A and a proposal program 23C. The CPU 20 reads out the medical care support program 23A and the proposal program 23C from the storage unit 22, develops the programs in the memory 21, and executes the developed medical care support programs 23A and proposal program 23C.

The storage unit 22 according to the present embodiment stores electronic medical record information 23B. The electronic medical record information 23B includes medical care information 25A and registration information 25B. The medical care information 25A is information used for medical care for the subject and information described in a so-called medical record, and includes a lot of personal information of the subject. The medical care information 25A according to the present embodiment is an example of first information of the present disclosure. The registration information 25B is information representing whether or not relative health information (details will be described below) relating to health of a relative of the subject is registered in the medical care support system 1.

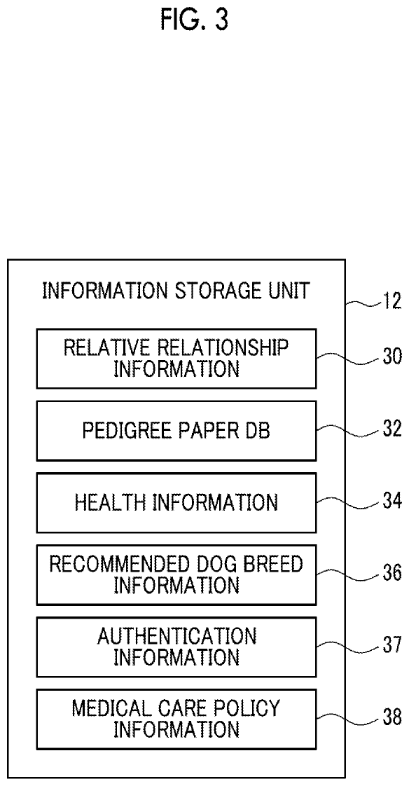

On the other hand, the information storage unit 12 according to the present embodiment is a cloud-type external storage that can be used so-called online. As shown in FIG. 3, the information storage unit 12 stores relative relationship information 30, a pedigree database (DB) 32, health information 34, recommended dog breed information 36, authentication information 37, and medical care policy information 38. In the present embodiment, a form in which the information storage unit 12 provided in the medical care support system 1 is one is described. However, the number of the information storage units 12 provided in the medical care support system 1 is not limited to one. For example, the medical care support system 1 comprises a plurality of information storage units 12, and the relative relationship information 30, the pedigree DB 32, the health information 34, the recommended dog breed information 36, the authentication information 37, and the medical care policy information 38 may be respectively stored in the separate information storage units 12. For example, there may be an aspect in which the medical care support system 1 comprises two information storage units 12 of an information storage unit 12 that stores the pedigree DB 32 and an information storage unit 12 that stores the relative relationship information 30, the health information 34, the recommended dog breed information 36, the authentication information 37, and the medical care policy information 38.

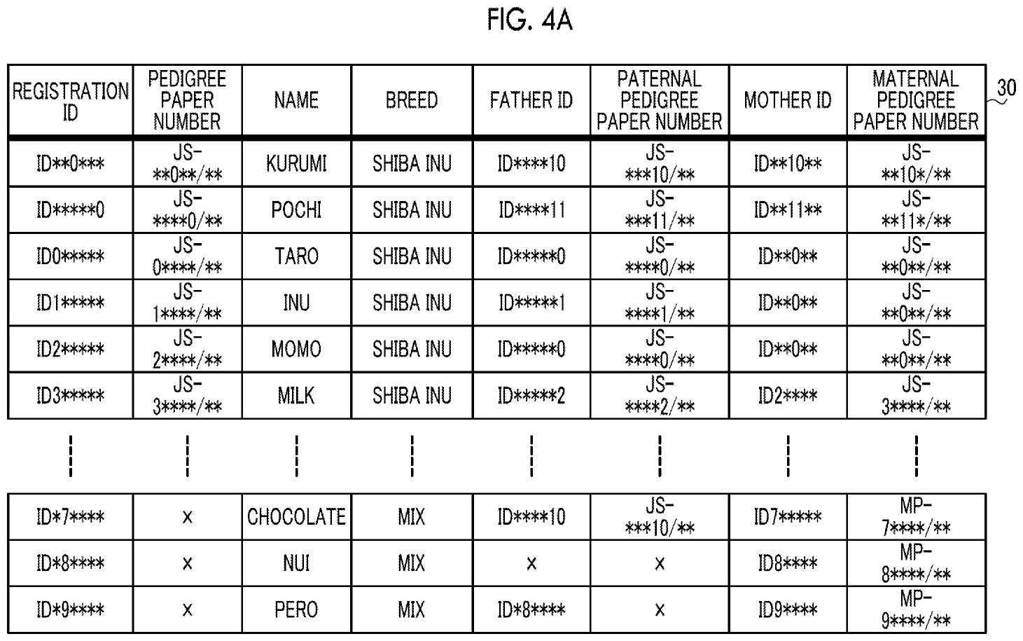

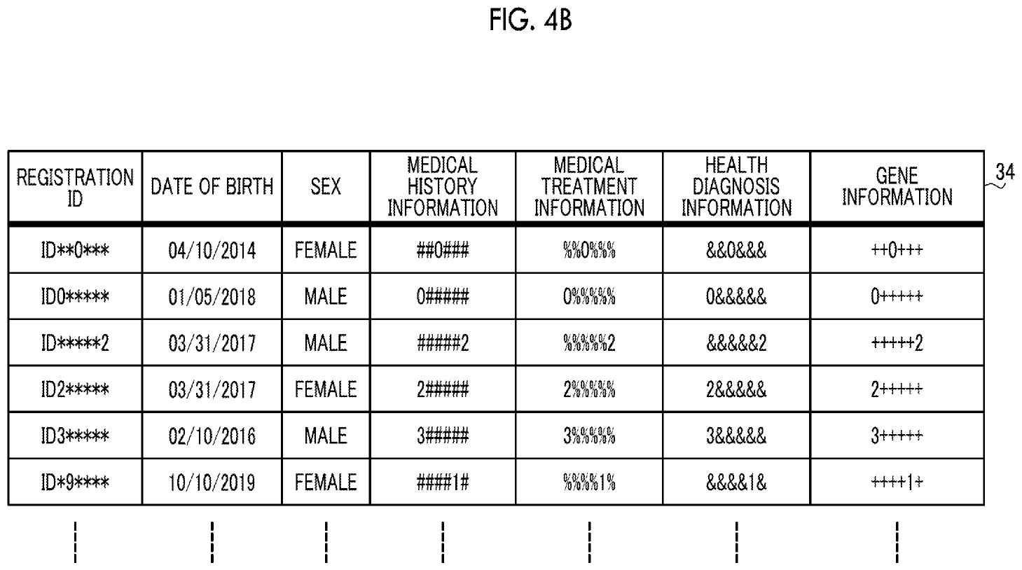

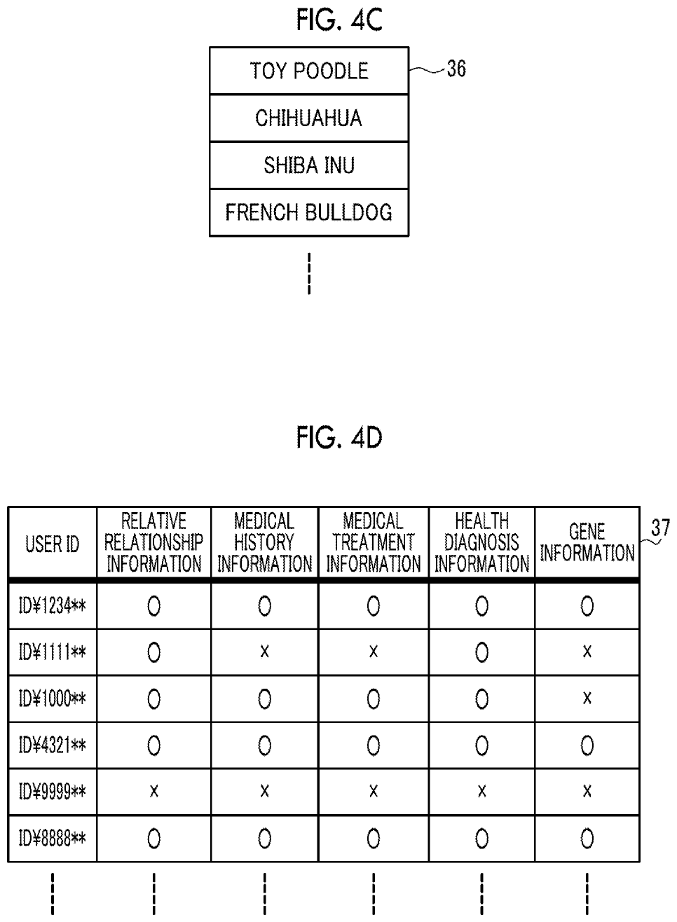

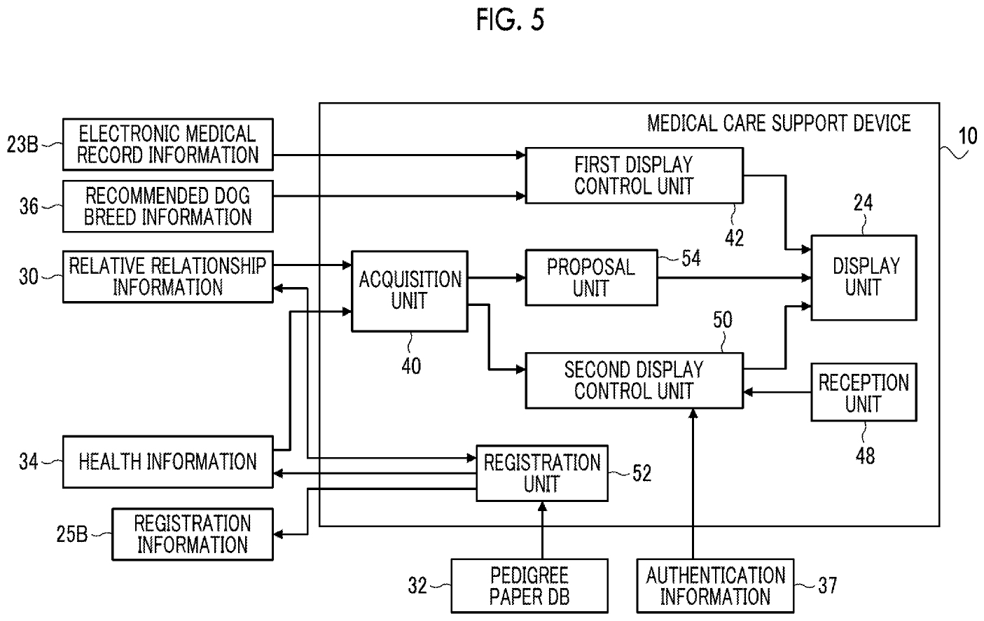

First, the relative relationship information 30 will be described. FIG. 4A shows an example of the relative relationship information 30. The relative relationship information 30 is information representing a relative relationship of the subject. In the present embodiment, parents (first-degree relative) of the subject are stored in correspondence with the subject as an example of the relative relationship. The relative relationship of the subject is not limited to the present embodiment and may be, for example, relatives (marriage and blood) within the third degree.

As a specific example, the relative relationship information 30 according to the present embodiment includes information on “registration ID”, “pedigree paper number”, “name”, “breed”, “father ID”, “paternal pedigree paper number”, “mother ID”, and “maternal pedigree paper number”.

The “registration ID” is an identifier (ID) for identifying a subject for which the relative health information is registered and identification information assigned to each of all subjects for which the relative relationship information 30 is registered in the present embodiment. In the present embodiment, the “registration ID” is different from “patient ID” such as an electronic medical record number assigned for identifying a subject at each animal hospital.

The “pedigree paper number” refers to a registration number of the subject in a case where the subject has a pedigree certificate, so-called pedigree paper. In a case where the subject does not have the pedigree paper, such as in a case where the subject is a hybrid such as a so-called mixed dog, the information corresponding to the “pedigree paper number” is not stored. In the example of the relative relationship information 30 shown in FIG. 4A, a subject to which “ID*7****” is assigned, a subject to which “ID*8****” is assigned, and a subject to which “ID*9****” is assigned do not have the pedigree paper, and an X mark representing that no information is stored is shown in FIG. 4A.

The “name” is a name of the subject itself. The “breed” represents a breed of the subject and thus represents a dog breed since the subject is a dog in the embodiment. In the embodiment, the “breed” includes a concept of species such as “dog” and “cat” in addition to the breed such as “dog breed”.

The “father ID” is a registration ID assigned to a father of the subject. As described above, the registration ID is assigned to all subjects for which the relative health information is registered in the medical care support system 1. Therefore, in a case where the relative health information of the father of the subject is not registered in the medical care support system 1, the father does not have the registration ID and thus the “father ID” is not stored. In the example of the relative relationship information 30 shown in FIG. 4A, a father of the subject to which “ID*8****” is assigned does not have the relative health information registered in the medical care support system 1. Therefore, the information corresponding to the “father ID” is not stored, and an X mark representing that no information is stored is shown in FIG. 4A.

The “paternal pedigree paper number” refers to a registration number of the father of the subject in a case where the father of the subject has the pedigree paper. In a case where the father of the subject does not have the pedigree paper, the information corresponding to the “paternal pedigree paper number” is not stored. In the example of the relative relationship information 30 shown in FIG. 4A, the father of the subject to which “ID*8****” is assigned and a father of the subject to which “ID*9****” is assigned do not have the pedigree paper, and an X mark representing that no information is stored is shown in FIG. 4A.

The “mother ID” is a registration ID assigned to a mother of the subject and is treated in the same manner as the “father ID”. The “maternal pedigree paper number” is a registration number of the subject in a case where the mother of the subject has the pedigree paper and is treated in the same manner as the “paternal pedigree paper number”.

As described above, the relative relationship information 30 according to the present embodiment stores information representing the relative relationship of the subject regardless of whether or not the subject and the parents of the subject have the pedigree papers.

Next, the pedigree DB 32 will be described. The pedigree DB 32 is a database of items described in the pedigree paper. With permission of an organization that issues the pedigree paper, a breeder, a store, and an owner of the subject, the items described in the pedigree paper are registered in the pedigree DB 32 in advance by an administrator or the like of the medical care support system 1. An individual to be registered in the pedigree DB 32 is not limited to an individual already registered in the medical care support system 1 (relative relationship information 30). Although not all the items described in the pedigree paper may be registered in the pedigree DB 32, items to be registered preferably include the dog breed, the registration number, sex, date of birth, information on parents, and information on grandparents, and information on great-grandparents, and more preferably include at least the registration number and the information on parents.

Next, the health information 34 will be described. FIG. 4B shows an example of the health information 34. The health information 34 is information on health states, from the past to the present, of all subjects (dogs in the present embodiment) registered in the medical care support system 1. More specifically, the health information 34 refers to the information on health states, from the past to the present, of all subjects registered in the medical care support system 1 from each of all medical care support devices 10 (refer to FIG. 1) provided in the medical care support system 1. As an example, the health information 34 according to the present embodiment includes “registration ID”, “date of birth”, “sex”, “medical history information”, “medical treatment information”, “health diagnosis information”, and “gene information”.

The “date of birth” is a date of birth of the subject itself, and the “sex” is a sex of the subject itself.

The “medical history information” is information on a disease that the subject is suffered from the past to the present. The “medical history information” includes information due to an external factor such as a fracture or a burn due to an accident in addition to the disease. The “medical treatment information” is information on medical treatment performed for a disease that the subject is suffered, an external wound such as an injury or the fracture, and the like, and includes information on an examination item or medication, for example. The “medical history information” and the “medical treatment information” generally form a pair from a viewpoint of medical treatment for a disease. Therefore, both pieces of information may be combined and treated as one piece of information. The “health diagnosis information” is information representing a result of a health diagnosis for the subject. The “gene information” preferably includes at least information on a genetic disease of the subject and includes gene examination information representing the presence or absence of a gene causing the genetic disease.

Detailed information of the “medical history information”, the “medical treatment information”, the “health diagnosis information”, and the “gene information” may be stored in the health information 34, or a providing destination of the detailed information, for example, a link destination where a content corresponding to the electronic medical record of the veterinarian can be referred may be stored.

In the present embodiment, the information is related to the health of the relative of the subject. Therefore, the relative relationship information 30 and the health information 34 (medical history information, medical treatment information, health diagnosis information, and gene information) are collectively referred to as the “relative health Information”.

Next, the recommended dog breed information 36 will be described. FIG. 4C shows an example of the recommended dog breed information 36. The recommended dog breed information 36 is information representing a dog breed for which the browsing of the relative health information is recommended. In the present embodiment, the dog breed for which the browsing thereof is recommended is a dog breed having a high possibility of developing the genetic disease. An example of the dog breed having a high possibility of developing the genetic disease includes a dog breed that is popular in at least one of a breeding number or a sales number. A dog breed with a large number of breeding or sales is likely to be inbred and is more likely to have a gene that causes the genetic disease. Therefore, the dog breed having a high possibility of developing the genetic disease is employed in the present embodiment. For the breeding number and the sales number, a breed within a predetermined higher order in ranking of at least one of the dog breeding number or the sales number is registered in the recommended dog breed information 36 in advance by the administrator or the like of the medical care support system 1, based on a result of a national survey on an actual situation of dog and cat breeding published by Japan Pet Food Association. The dog breed having a high possibility of developing the genetic disease is not limited to the present embodiment and may be, for example, a dog breed known to have a high incidence of the genetic disease or a dog breed known to have a high incidence of a serious genetic disease.

Next, the authentication information 37 will be described. FIG. 4D shows an example of the authentication information 37. The authentication information 37 is information representing a user who is permitted (authenticated) to browse the relative relationship information 30 and the medical history information, the medical treatment information, the health diagnosis information, and the gene information included in the health information 34 among the relative health information. As shown in FIG. 4D, a user ID assigned to the veterinarian, an animal nurse, or the like for identifying each individual is in correspondence with information representing whether the browsing is permitted or not permitted for each piece of the above information. As an example, in the present embodiment, a user who is not permitted to browse the relative relationship information 30 is not permitted (authenticated) also to browse the medical history information, the medical treatment information, the health diagnosis information, and the gene information. In the example of FIG. 4D, a mark “∘” is shown in a case representing that the browsing is permitted, and a mark “X” is shown in a case representing that the browsing is not permitted. The information representing permission and non-permission of the browsing of each piece of information is registered in the authentication information 37 by the administrator or the like of the medical care support system 1.

Next, the medical care policy information 38 will be described. The medical care policy information 38 is stored, for each predetermined disease type, in correspondence with a medical care policy, such as information for prevention of the disease, a required examination, a medical treatment policy in a case where the subject is suffered, and a type of medication.

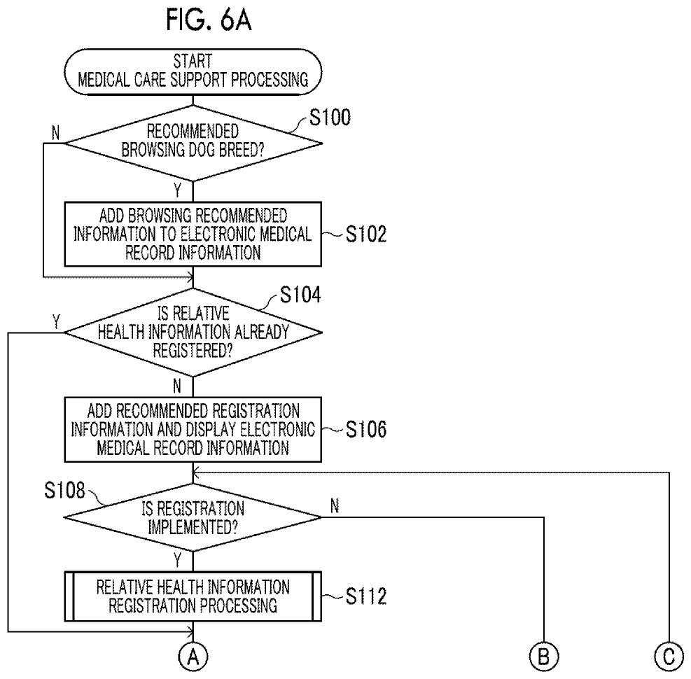

Next, a functional configuration of the medical care support device 10 according to the present embodiment will be described with reference to FIG. 5. FIG. 5 is a functional block diagram representing an example of the configuration of the medical care support device 10 according to the present embodiment.

As shown in FIG. 5, the medical care support device 10 comprises an acquisition unit 40, a first display control unit 42, a reception unit 48, a second display control unit 50, a registration unit 52, and a proposal unit 54. The CPU 20 of the medical care support device 10 executes the medical care support program 23A to function as the acquisition unit 40, the first display control unit 42, the reception unit 48, the second display control unit 50, and the registration unit 52. The CPU 20 of the medical care support device 10 executes the proposal program 23C to function as the proposal unit 54.

The acquisition unit 40 acquires the relative relationship information 30 and the health information 34 from the information storage unit 12 and outputs the acquired information to the second display control unit 50 and the proposal unit 54.

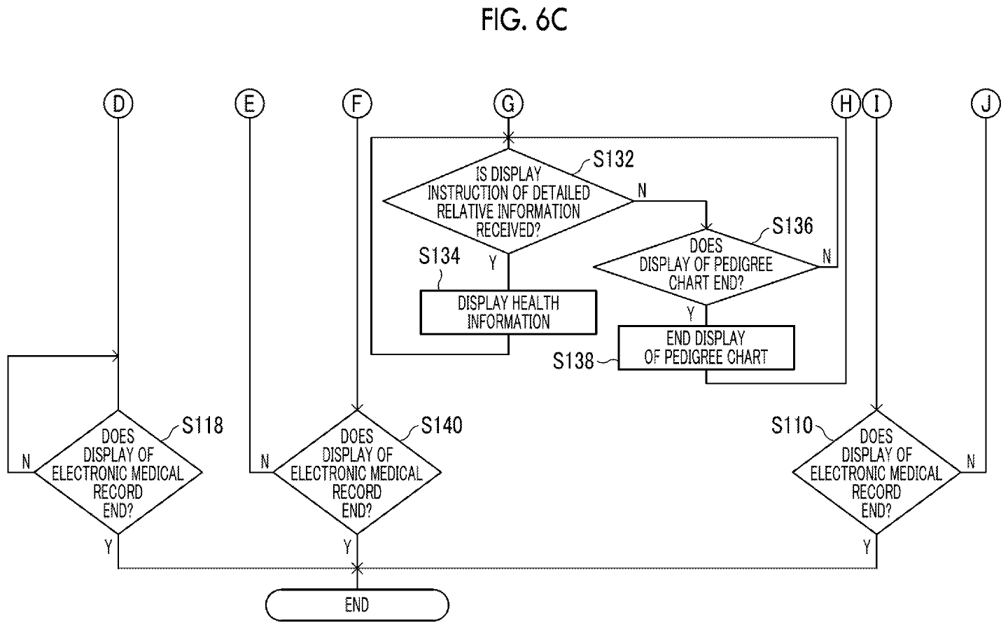

The first display control unit 42 performs control to acquire the electronic medical record information 23B from the storage unit 22 and display the acquired electronic medical record information 23B on the display unit 24. The first display control unit 42 performs control to acquire the recommended dog breed information 36 from the information storage unit 12 and display browsing recommended information 60 (refer to FIG. 7), on the display unit 24, representing that the browsing is recommended in a case where a dog breed of the subject is the dog breed stored in the recommended dog breed information 36, that is, the dog breed for which the browsing of the relative health information is recommended. The first display control unit 42 performs control to display recommended registration information 62 (refer to FIG. 7), on the display unit 24, that recommends the registration of the relative health information in a case where the relative health information of the subject is not registered, based on the registration information 25B included in the electronic medical record information 23B. The recommended registration information 62 according to the present embodiment is an example of recommended storage information of the present disclosure.

The reception unit 48 receives a display instruction input by the user through the input unit 26 to display the relative health information on the display unit 24 and outputs information representing that the display instruction is received to the second display control unit 50.

The second display control unit 50 performs control to display relative health information, on the display unit 24, that is permitted to be browsed, based on the relative relationship information 30 and the health information 34 acquired by the acquisition unit 40 with reference to the authentication information 37 in a case where the information representing that the display instruction is received is input from the reception unit 48.

The registration unit 52 performs processing of registering the relative health information of the subject in the relative relationship information 30 and the health information 34 with reference to the pedigree DB 32 and the like. The registration unit 52 stores information representing that the relative health information is registered in the registration information 25B in a case where the relative health information of the subject is registered in the relative relationship information 30 and the health information 34.

The proposal unit 54 displays information, on the display unit 24, that proposes at least one of the examination or the medical care policy for the subject based on the relative health information with reference to the relative relationship information 30 and the health information 34 acquired by the acquisition unit 40.

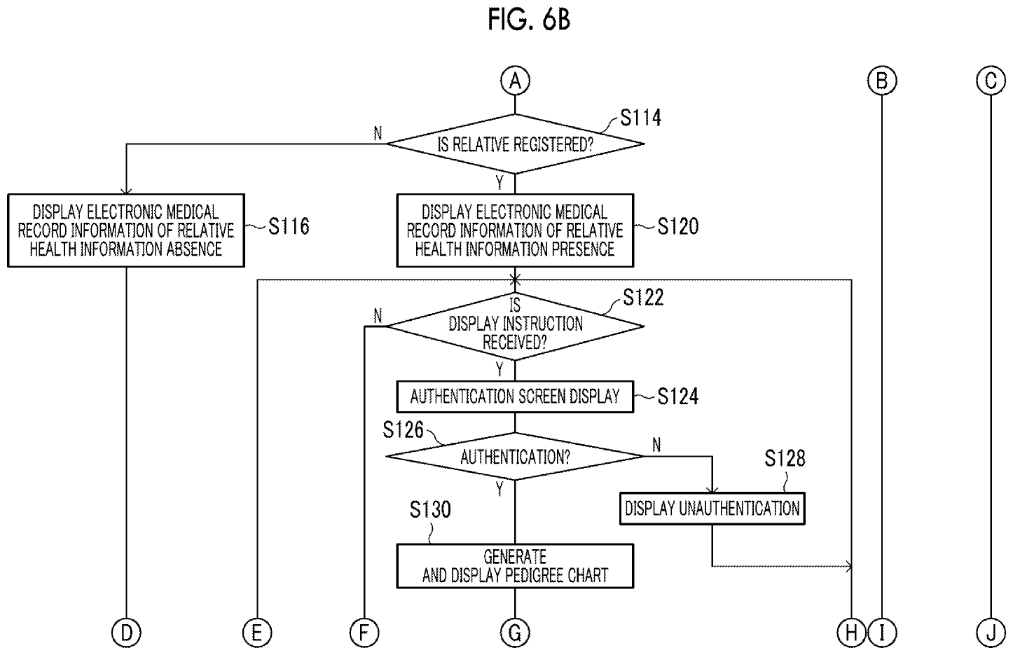

Next, an action of the medical care support device 10 according to the present embodiment will be described. First, medical care support processing in the medical care support device 10 will be described. The CPU 20 executes the medical care support program 23A to execute the medical care support processing shown in FIG. 6. The medical care support processing shown in FIGS. 6A to 6C is executed in a case where an instruction to display the electronic medical record of the subject is input through the input unit 26 by the user.

In Step S100 shown in FIG. 6A, the first display control unit 42 determines whether or not a dog breed of the subject is the dog breed for which the browsing of the relative health information is recommended. Specifically, the first display control unit 42 specifies the dog breed of the subject from the medical care information 25A of the electronic medical record information 23B to determine whether or not the specified dog breed of the subject is a dog breed registered in the recommended dog breed information 36. In a case where the dog breed of the subject is not the dog breed registered in the recommended dog breed information 36, the determination in Step S100 is negative and the processing proceeds to Step S104. On the other hand, in a case where the dog breed of the subject is the dog breed registered in the recommended dog breed information 36, the determination in Step S100 is affirmative and the processing proceeds to Step S102.

In Step S102, the first display control unit 42 adds the browsing recommended information 60 (refer to FIG. 7) that recommends the browsing of the relative health information of the subject to the electronic medical record information 23B.

In next Step S104, the first display control unit 42 determines whether or not the relative health information of the subject is registered. Specifically, the first display control unit 42 determines that the relative health information of the subject is already registered in a case where the information representing that the relative health information is registered in the registration information 25B is stored. In a case where the relative health information of the subject is already registered, the determination in Step S104 is affirmative and the processing proceeds to Step S114. On the other hand, in a case where the relative health information of the subject is not registered, the determination in Step S104 is negative and the processing proceeds to Step S106.

In Step S106, the first display control unit 42 performs control of displaying the electronic medical record information 23B, on the display unit 24, to which the recommended registration information 62 (refer to FIG. 7) that recommends the registration of the relative health information of the subject itself is added. FIG. 7 shows an example of the electronic medical record information 23B which is displayed on the display unit 24 by the first display control unit 42 and to which the browsing recommended information 60 and the recommended registration information 62 are added.

As shown in FIG. 7, the medical care information 25A including patient information 64A, owner information 64B, medical examination information 64C, vaccine information 64D, medical care information 64E, meal information 64F, medication information 64G, message 64H, and order information 641 is displayed on the display unit 24 as the electronic medical record information 23B.

The patient information 64A is information on a patient, that is, individual pets which are subjects.

The owner information 64B is information on the owner of the subject. The medical examination information 64C is information on a medical examination that the subject is already received. The vaccine information 64D is information on a vaccine that the subject is already inoculated. The medical care information 64E is information on the medical care for the subject from the past to the present and includes also a health diagnosis result in the present embodiment. The medical care information 64E shown in FIG. 7 shows a state where a first page of a plurality of pages is displayed on the display unit 24. The meal information 64F is information on an eating habit of the subject. The medication information 64G is information on medication to the subject. The message 64H is information obtained by the user observing the subject itself, information heard from the owner of the subject, or the like in the medical care and is information entered by the user using the input unit 26. The order information 641 is information entered by the user using the input unit 26 in a case where the examination or the like for the subject is ordered.

The browsing recommended information 60 and the recommended registration information 62 are displayed in a superimposed manner on the patient information 64A in a display area where the patient information 64A is displayed. The recommended registration information 62 to be displayed on the display unit 24 is an example of second information of the present disclosure in a case representing that there is no relative health information.

In next Step S108, the registration unit 52 determines whether or not to register the relative health information. As an example, in the present embodiment, the display area of the recommended registration information 62 on the display unit 24 is an instruction button for performing an instruction to implement the registration of the relative health information. The user such as the veterinarian or the animal nurse clicks the display area of the recommended registration information 62 with the input unit 26 in a case where the relative health information of the subject is registered. In a case where the display area of the recommended registration information 62 is clicked, the registration unit 52 determines that the registration of the relative health information is implemented. In a case where the display area of the recommended registration information 62 is not clicked by the user, the determination in Step S108 is negative and the processing proceeds to Step S110.

In Step S110, the registration unit 52 determines whether or not to end the display of the electronic medical record. The medical care support device 10 according to the present embodiment continues to display the electronic medical record on the display unit 24 until the user inputs an instruction to end the display of the electronic medical record through the input unit 26, and ends the display of the electronic medical record on the display unit 24 in a case where the instruction to end the display of the electronic medical record is input. Therefore, in a case where the instruction to end the display of the electronic medical record is input, the determination in Step S110 is affirmative and the medical care support processing ends. On the other hand, in a case where the instruction to end the display of the electronic medical record is not input, the determination in Step S110 is negative and the processing returns to Step S108.

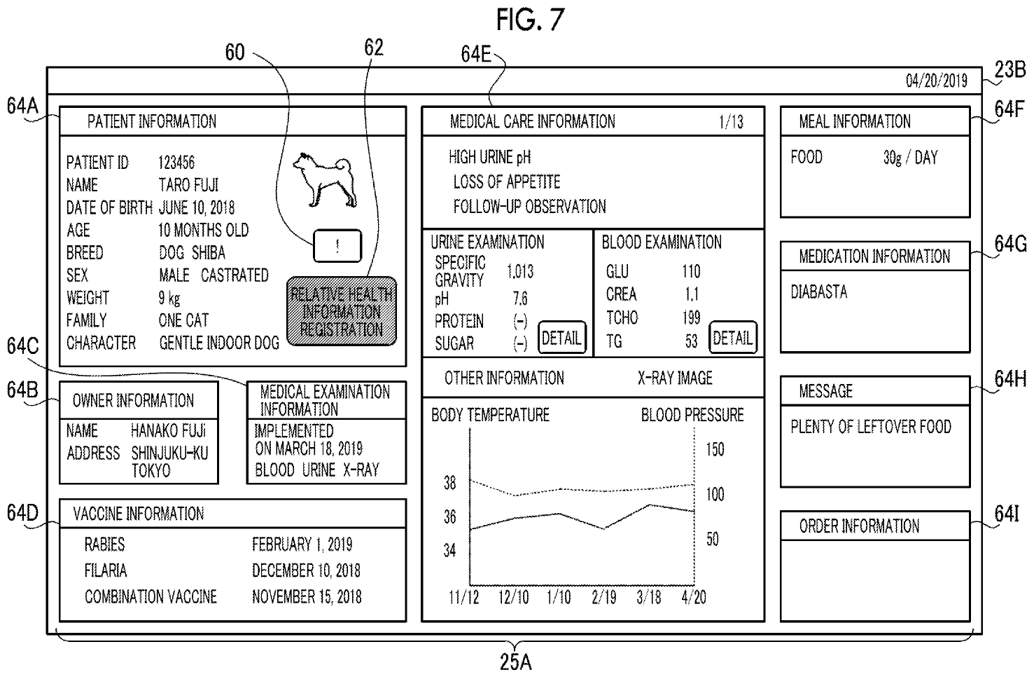

On the other hand, in a case where the display area of the recommended registration information 62 is clicked by the user, the determination in Step S108 is affirmative and the processing proceeds to Step S112 to implement relative health information registration processing shown in FIG. 8 as an example and to register the relative health information of the subject in the medical care support system 1.

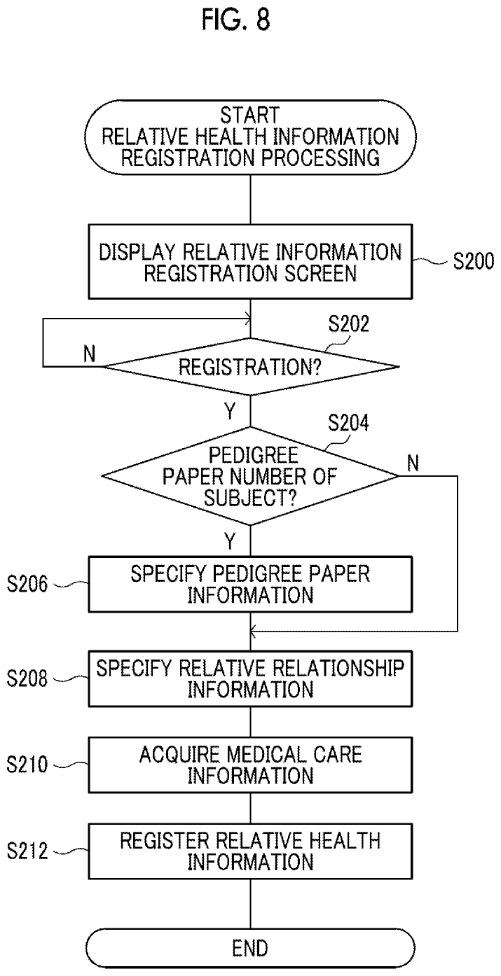

In Step S200 in FIG. 8, the registration unit 52 displays a relative health information registration screen 66 shown in FIG. 9 as an example on the display unit 24. The registration unit 52 may display the relative health information registration screen 66 in a state where at least a part of the screen is superimposed on the electronic medical record information 23B, on the display area of the display unit 24 where the electronic medical record information 23B is displayed. For example, the registration unit 52 may display the relative health information registration screen 66 instead of the electronic medical record information 23B or in a display area different from the display area where the electronic medical record information 23B is displayed.

The relative health information registration screen 66 shown in FIG. 9 is an example of a screen for the user to input information for registering the relative health information. FIG. 9 shows a state where the pedigree paper number of the subject itself is input by the user.

As an example, in the present embodiment, in a case where the relative health information is newly registered, the patient ID is required to be input. Even in a case where only the patient ID of the subject itself is input, the relative health information of the subject can be registered in the medical care support system 1. In the case, the relative health information to be registered does not include the relative relationship information 30 of the parents of the subject.

In the present embodiment, the user inputs at least one of the pedigree paper number of the subject, the mother, or the father or at least one of the registration ID of the mother or the father on the relative health information registration screen 66 through the input unit 26, and thus the relative health information can be registered in the medical care support system 1. In the present embodiment, in a case where the relative health information of at least one of the mother or the father of the subject is already registered in the medical care support system 1, the user inputs registration IDs of the registered parents, and thus the relative health information including the relative relationship information 30 of the parents can be registered even in a case where the subject does not have the pedigree paper.

After inputting the patient ID and the pedigree paper number or the registration ID, the user clicks a registration button 67 in the relative health information registration screen 66 with the input unit 26. In next Step S202, the registration unit 52 determines whether or not the registration button 67 is clicked. The determination in Step S202 is negative until the registration button 67 is clicked and the determination in Step S202 is affirmative in a case where the registration button 67 is clicked, and the processing proceeds to Step S204.

In Step S204, the registration unit 52 determines whether or not the pedigree paper number of the subject is input on the relative health information registration screen 66. In a case where the pedigree paper number of the subject is not input on the relative health information registration screen 66, the determination in Step S204 is negative and the processing proceeds to Step S208.

On the other hand, in a case where the pedigree paper number of the subject is input on the relative health information registration screen 66, the determination in Step S204 is affirmative and the processing proceeds to Step S206.

In Step S206, the registration unit 52 specifies pedigree paper information corresponding to the input pedigree paper number from the pedigree DB 32. Specifically, the registration unit 52 specifies pedigree paper numbers of parents corresponding to a dog with the input pedigree paper number from the pedigree DB 32.

In next Step S208, the registration unit 52 specifies relative relationship information. Specifically, the registration unit 52 specifies a pedigree paper number corresponding to the input registration ID from the relative relationship information 30 in a case where at least one of the registration IDs of the parents of the subject is input on the relative health information registration screen 66. In a case where the pedigree paper number is not stored in the relative relationship information 30, the pedigree paper number is not specified.

The registration unit 52 specifies a registration ID corresponding to the input pedigree paper number from the relative relationship information 30 in a case where at least one of the pedigree paper numbers of the parents of the subject is input on the relative health information registration screen 66. The registration unit 52 specifies a registration ID corresponding to respective pedigree paper numbers of the parents specified in Step S206 from the relative relationship information 30 in a case where the pedigree paper number of the subject is input on the relative health information registration screen 66, that is, in a case where the processing proceeds to Step S208 after Step S206. In any case, in a case where the relative health information of the parents whose pedigree paper numbers are specified is not registered in the medical care support system 1, the registration ID is not specified.

In next Step S210, the registration unit 52 acquires the medical care information 25A. Specifically, the registration unit 52 acquires the name, breed, date of birth, sex, medical treatment information, health diagnosis information, and gene information of the subject included in the medical care information 25A based on the input patient ID.

In next Step S212, the registration unit 52 assigns a registration ID to the subject, registers the relative health information in the relative relationship information 30 and the health information 34, then ends the relative health information registration processing, and the processing proceeds to Step S114 of the medical care support processing shown in FIG. 6B.

In Step S114, the first display control unit 42 determines whether or not the relative health information of the relative of the subject is registered in the medical care support system 1. For example, in a case where the relative health information of the subject is registered in the medical care support system 1 for the first time in relatives to which the subject belongs, the determination in Step S114 is negative and the processing proceeds to Step S116.

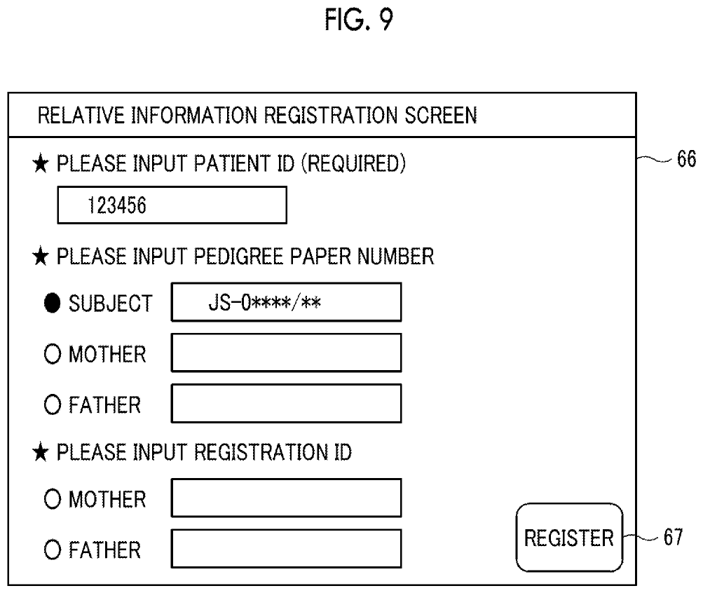

In Step S116, the first display control unit 42 performs control of displaying the electronic medical record information 23B, on the display unit 24, to which relative health information absence information 68 (refer to FIG. 10) representing that there is no relative health information is added. FIG. 10 shows an example of the electronic medical record information 23B to which the relative health information absence information 68 is added, which is displayed on the display unit 24 by the first display control unit 42. As shown in FIG. 10, the browsing recommended information 60 and the relative health information absence information 68 are displayed in the display area where the patient information 64A is displayed. The relative health information absence information 68 according to the present embodiment is an example of the second information of the present disclosure in the case representing that there is no relative health information.

In next Step S118, the first display control unit 42 determines whether or not to end the display of the electronic medical record, as in Step S110. The determination in Step S118 is negative until the instruction to end the display of the electronic medical record is input. On the other hand, in a case where the instruction to end the display of the electronic medical record is input, the determination in Step S118 is affirmative and the medical care support processing ends.

On the other hand, in a case where the relative health information of the relative of the subject is registered in the medical care support system 1 in Step S114, the determination in Step S114 is affirmative and the processing proceeds to Step S120.

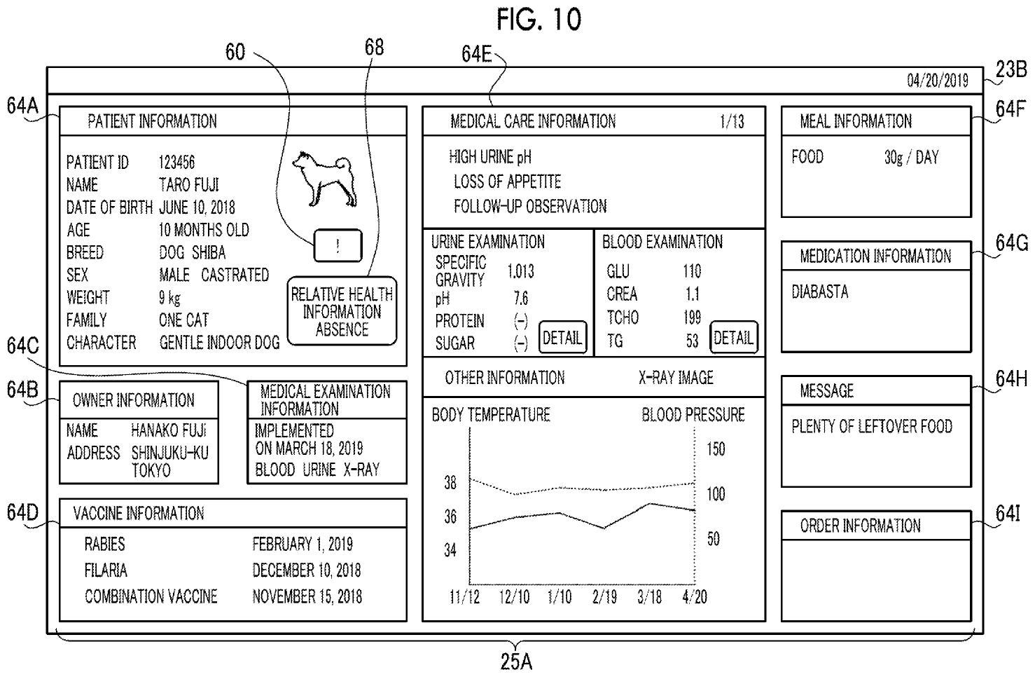

In Step S120, the first display control unit 42 performs control of displaying the electronic medical record information 23B, on the display unit 24, to which relative health information presence information 70 (refer to FIG. 11) representing that there is relative health information is added. FIG. 11 shows an example of the electronic medical record information 23B to which the relative health information presence information 70 is added, which is displayed on the display unit 24 by the first display control unit 42. As shown in FIG. 11, the browsing recommended information 60 and the relative health information presence information 70 are displayed in the display area where the patient information 64A is displayed. The relative health information presence information 70 to be displayed on the display unit 24 according to the present embodiment is an example of the second information of the present disclosure in the case representing that there is relative health information.

In next Step S122, the reception unit 48 determines whether or not an instruction to display the relative health information (hereinafter referred to as “display instruction”) is received. As an example, in the present embodiment, the display area of the relative health information presence information 70 on the display unit 24 is an instruction button for performing an instruction to display the relative health information by the user. In a case where the instruction to display the relative health information of the relative of the subject is performed, the user clicks the display area of the relative health information presence information 70 with the input unit 26. The reception unit 48 determines that the display instruction is received in a case where the display area of the relative health information presence information 70 is clicked. In a case where the display area of the relative health information presence information 70 is clicked, the determination in Step S122 is affirmative and the processing proceeds to Step S124.

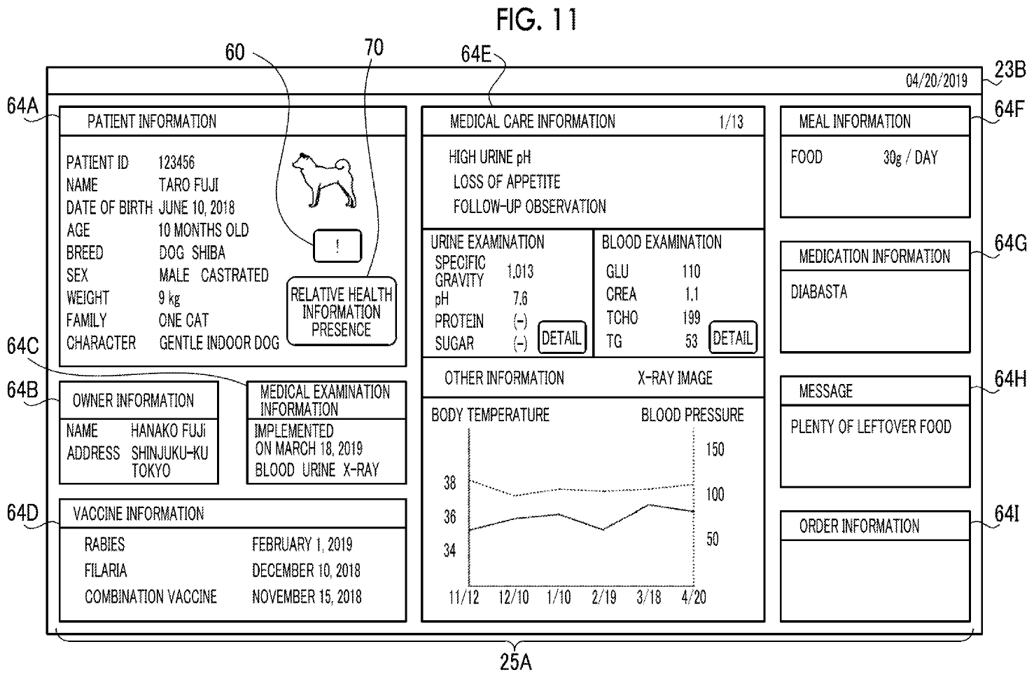

In Step S124, the reception unit 48 displays an authentication screen 72 shown in FIG. 12 as an example on the display unit 24. The reception unit 48 may display the authentication screen 72 in a state where at least a part of the screen is superimposed on the electronic medical record information 23B, on the display area of the display unit 24 where the electronic medical record information 23B is displayed. For example, the reception unit 48 may display the authentication screen 72 instead of the electronic medical record information 23B or in a display area different from the display area where the electronic medical record information 23B is displayed.

The authentication screen 72 shown in FIG. 12 is an example of a screen for inputting a user ID of a user who browses the relative health information. After inputting the user ID, the user clicks a confirm button 73 in the authentication screen 72 with the input unit 26.

In next Step S126, the second display control unit 50 determines whether or not to authenticate a user corresponding to the input user ID. Specifically, the second display control unit 50 determines whether or not the browsing of the relative relationship information 30 is permitted for the input user ID with reference to the authentication information 37. In a case where the browsing of the relative relationship information 30 is not permitted, the user is not authenticated. Therefore, the determination in Step S126 is negative and the processing proceeds to Step S128. In Step S128, the second display control unit 50 displays information, on the display unit 24, representing that the authentication is not possible, and then the processing returns to Step S122.

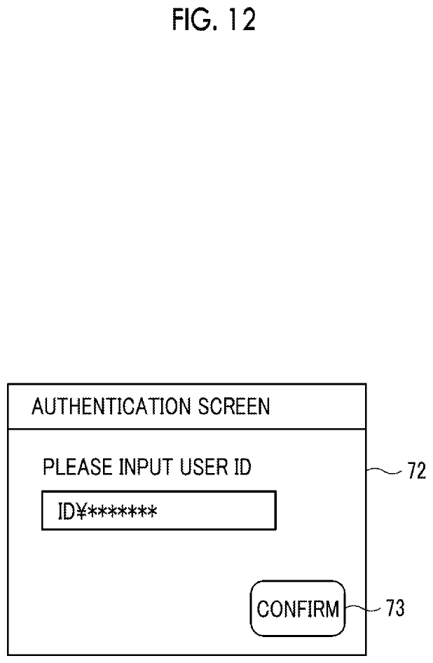

On the other hand, in a case where the browsing of the relative relationship information 30 is permitted, the determination in Step S126 is affirmative to authenticate the user and the processing proceeds to Step S130. In Step S130, the second display control unit 50 performs control of generating a pedigree chart of the subject and displaying the generated chart on the display unit 24 with reference to the relative relationship information 30. FIG. 13 shows an example of a pedigree chart 76 to be displayed on the display unit 24. The pedigree chart 76 shown in FIG. 13 displays a photograph 77 of each of the relatives including the subject itself, a name 78 of each relative, and personal information 79 indicating the subject itself. The pedigree chart 76 shown in FIG. 13 is an example of the pedigree chart in a case where the subject is a Shiba Inu named “Taro”, which is shown in FIG. 11 as an example of the electronic medical record information 23B. A method of generating the pedigree chart 76 by the second display control unit 50 in the case will be specifically described.

The second display control unit 50 specifies a father ID “ID*****0” and a mother ID “ID**0***” corresponding to the subject “Taro” (subject ID0*****) with reference to the relative relationship information 30. The second display control unit 50 specifies “Pochi”, as the father, with the specified father ID of the “Taro” as the registration ID. The second display control unit 50 specifies “Momo” whose father ID is “ID*****0” and whose registration ID is “ID2*****”, from the relative relationship information 30, as a sibling having the same father. A mother ID of “Momo” is the same “ID**0***” as “Taro”. Therefore, “Momo” is specified as the sibling of “Taro” having the same parents. The second display control unit 50 specifies “Milk” whose registration ID in correspondence with the registration ID of “Momo” as the mother ID is “ID3*****” as a nephew and also specifies a father ID of “Milk”.

On the other hand, the second display control unit 50 specifies “Kurumi” with the specified mother ID as the registration ID as a mother of “Taro”. The second display control unit 50 specifies “Inu” with the registration ID of “Kurumi” as the mother ID and whose registration ID is “ID1*****”, from the relative relationship information 30, as a sibling having the same mother and different fathers and also specifies a father ID of “Inu”.

The second display control unit 50 generates the pedigree chart 76 representing the specified relative relationship. As described above, the father ID, the mother ID, and the registration ID are traced, and thus it is possible to generate the pedigree chart 76 including the first-degree relative or higher even in a case where only first-degree relative is in correspondence with the registration ID in the relative relationship information 30.

In a case where the electronic medical record information 23B includes a photograph of the patient (subject), the included photograph may be used as the photograph 77 used for the pedigree chart 76. In a case where the electronic medical record information 23B does not include a photograph of the patient (subject), for example, information representing that there is no photograph may be displayed or a predetermined image that replaces the photograph may be displayed.

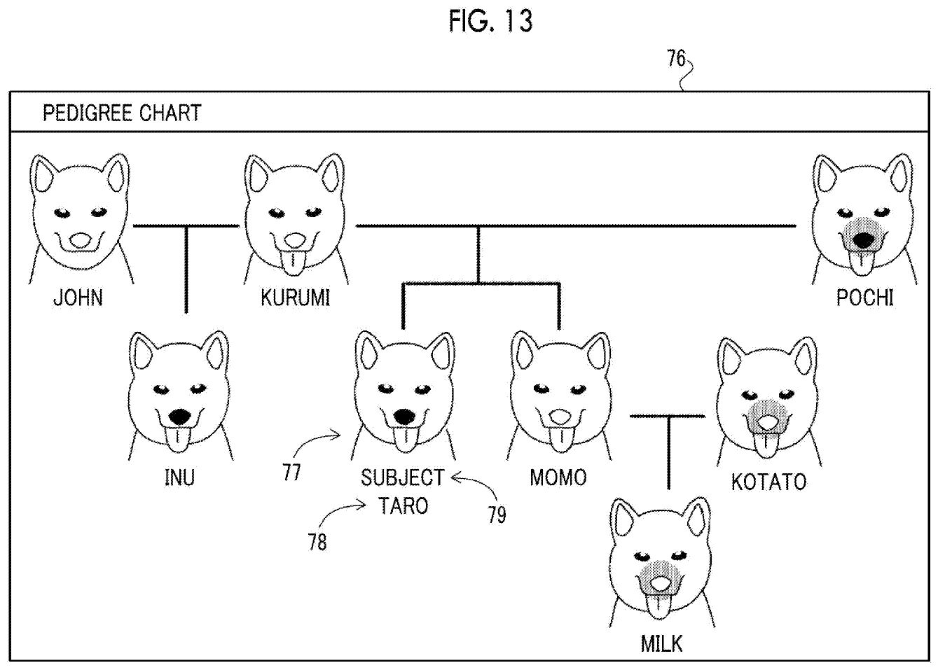

In next Step S132, the second display control unit 50 determines whether or not an instruction to display detailed relative information 82 shown in FIG. 14 as an example is received. In the present embodiment, in a case where the user clicks the photograph 77 of the pedigree chart 76 displayed on the display unit 24 with the input unit 26 to perform the display instruction, it is possible to display the detailed relative information 82 including a detail such as information on the health of the relative corresponding to the photograph 77 or personal information. In a case where the display instruction of the detailed relative information 82 is received, the determination in Step S132 is affirmative and the processing proceeds to Step S134.

In Step S134, the second display control unit 50 displays the detailed relative information 82 shown in FIG. 14 as an example on the display unit 24, and then the processing returns to Step S132. Specifically, the second display control unit 50 acquires information permitted to be browsed by the authentication information 37 for an authenticated user from the relative relationship information 30 and the health information 34 for the subject whose photograph 77 is clicked, generates the detailed relative information 82, and displays the generated information on the display unit 24. In the present embodiment, the detailed relative information 82 is displayed on the display unit 24 side by side with the pedigree chart 76, but a display method of the detailed relative information 82 is not particularly limited. For example, the second display control unit 50 may display the detailed relative information 82 in a superimposed manner on the pedigree chart 76.

The detailed relative information 82 shown in FIG. 14 includes “relationship” between the subject and a displayed relative, “name”, “registration ID”, “date of birth”, “age”, “breed”, “sex”, “medical history”, regular “hospital”, and presence or absence of “genetic disease”. A detail button 83 is displayed in the detailed relative information 82. The user clicks the detail button 83 to further display, on the display unit 24, the details of “medical history information”, “medical treatment information”, “health diagnosis information”, and “gene information” that are permitted to be browsed.

As described above, in the medical care support device 10 according to the present embodiment, detailed information on the health of the relative of the subject is sequentially displayed on the display unit 24 according to the permission of browsing every time the user performs the display instruction. Therefore, with the medical care support device 10 according to the present embodiment, information to be disclosed about the relative personal information can be further restricted and thus the personal information is further protected.

On the other hand, in a case where the display instruction of the detailed relative information 82 is not received in a state where the pedigree chart 76 is displayed on the display unit 24, the determination in Step S132 is negative and the processing proceeds to Step S136.

In Step S136, the second display control unit 50 determines whether or not to end the display of the pedigree chart 76. As an example, in the present embodiment, the user clicks the photograph 77 of the subject itself in the pedigree chart 76 with the input unit 26 to perform the instruction to end the display of the pedigree chart 76. In a case where the instruction to end the display of the pedigree chart 76 is not performed, the determination in Step S136 is negative and the process returns to Step S132. On the other hand, in a case where the instruction to end the display of the pedigree chart 76 is performed, the determination in Step S136 is affirmative and the processing proceeds to Step S138.

In Step S138, the second display control unit 50 ends the display of the pedigree chart 76, and then the processing returns to Step S122. In a case where the display of the pedigree chart 76 ends, there is a state where the electronic medical record information 23B to which the relative health information presence information 70 shown in FIG. 11 is added is displayed on the display unit 24.

In the displayed electronic medical record information 23B, in a case where the user does not click the area of the relative health information presence information 70, the determination in Step S122 is negative and the processing proceeds to Step S140.

In Step S140, the reception unit 48 determines whether or not to end the display of the electronic medical record, as in Step S110. In a case where an instruction to end the display of the electronic medical record is not input, the determination in Step S140 is negative and the process returns to Step S122. On the other hand, in a case where the instruction to end the display of the electronic medical record is input, the determination in Step S140 is affirmative and the medical care support processing ends.

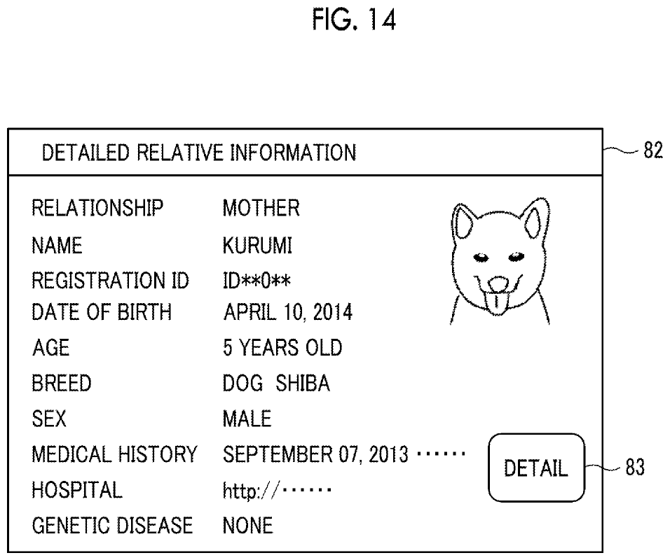

Next, proposal processing in the action of the medical care support device 10 according to the present embodiment will be described. The CPU 20 executes the proposal program 23C to execute the proposal processing shown in FIG. 15. As an example, the proposal processing shown in FIG. 15 according to the present embodiment is executed in a case where the user is authenticated in Step S126 of the above medical care support processing (refer to FIG. 6B).

In Step S300 of FIG. 15, the proposal unit 54 acquires the health information 34 of the relative of the subject. As an example, the proposal unit 54 according to the present embodiment acquires the health information 34 of all relatives registered in the medical care support system 1 among relatives within the third-degree relative to the subject.

In next Step S302, the proposal unit 54 counts, for each type of disease suffered by relatives, a count value according to the number of suffered relatives as suffering risk. In the present embodiment, a relative having a closer relative degree, in other words, having a closer blood relationship to the subject has a higher suffering risk value. The value of the suffering risk is higher as a probability to be suffered is higher and as there is a more severe disease with high mortality or the like. That is, the value of the suffering risk represents that the possibility that the subject suffers from the disease is higher as the value thereof is higher. The value of the suffering risk represents that there is a higher possibility that the subject dies in a case where the subject suffers from the disease and becomes more serious as the value thereof is higher. A type of disease for which the suffering risk is counted may be determined in advance or, for example, may be determined for each breed (dog breed).

In next Step S304, the proposal unit 54 determines whether or not the value of the suffering risk is equal to or larger than a threshold value (suffering risk □ threshold value). The threshold value may be set in the medical care support system 1 in advance by the administrator or the like of the medical care support system 1 or may be set by the user. The threshold value may be determined for each type of disease.

In a case where the suffering risk value is not equal to or larger than the threshold value, in other words, in a case where the suffering risk value is less than the threshold value, the determination in Step S304 is negative and the proposal processing ends. On the other hand, in a case where the suffering risk value is equal to or larger than the threshold value, in other words, in a case where the suffering risk value is equal to or larger than the threshold value, the determination in Step S304 is affirmative and the processing proceeds to Step S306.

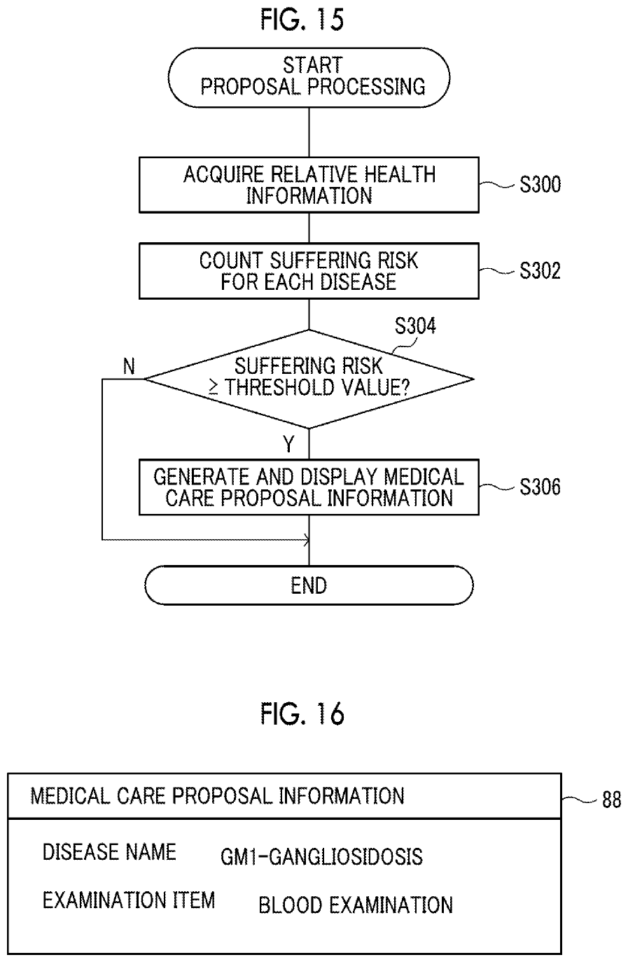

In Step S306, the proposal unit 54 generates medical care proposal information 88 shown in FIG. 16 as an example based on the medical care policy information 38, then displays the generated information on the display unit 24, and ends the proposal processing. As an example, the medical care proposal information 88 shown in FIG. 16 includes a “disease name” of a disease that may be suffered and an “examination item” for examining whether or not the subject suffers from the disease.

The medical care proposal information 88 may be displayed in a state where the information is superimposed on the electronic medical record information 23B displayed on the display unit 24. For example, the medical care proposal information 88 may be displayed in a state of being embedded in a part of the electronic medical record information 23B, such as being included in the order information 641. In the case, the medical care proposal information 88 is preferably included in the electronic medical record information 23B and stored in the information storage unit 12.

In the present embodiment, an example has been described in which the proposal unit 54 generates the medical care proposal information 88 based on the medical care policy information 38 stored in the information storage unit 12 in advance. However, the proposal unit 54 generates the medical care proposal information 88 from the health information 34 of the subject. For example, the proposal unit 54 may quantify and count a degree of effect on a medical treatment to generate information on the most effective medical treatment method as the medical care proposal information 88.

As described above, the information storage unit 12 according to the present embodiment can propose the medical care proposal information 88 based on the relative health information (health information 34) of the relative of the subject. Therefore, it is possible to propose an effective medical care policy suitable for the subject itself.

As described above, the medical care support device 10 according to the present embodiment includes the first display control unit 42 that performs the control of displaying, on the display unit 24, the electronic medical record information 23B including the medical care information 25A used for the medical care for the subject and the relative health information absence information 68 or the health information presence information 70 representing the presence or absence of the relative health information on the health of the relative of the subject, the reception unit 48 that receives the display instruction performed according to the display of the relative health information presence information 70 for displaying the relative health information on the display unit 24, and the second display control unit 50 that performs the control of displaying the relative health information on the display unit 24 in a case where the reception unit 48 receives the display instruction.

In a case where the relative health information of the relative is suddenly displayed on the electronic medical record, it may not be preferable from the viewpoint of personal information protection. For example, in a case where the subject is a pet and an owner of the pet which is the subject (hereinafter referred to as “subject owner”) and an owner of a pet which is a relative of the subject (hereinafter referred to as “relative owner”) are other persons, the relative owner may not want to disclose the relative health information of his/her pet to the subject owner. On the other hand, the relative health information of the relative of the subject is useful for the medical care for the subject. Therefore, the veterinarian may want to refer to the information and the relative owner may also disclose the relative health information to the veterinarian. However, in a case where the electronic medical record information 23B includes the relative health information of the relative, there is a high concern that not only the veterinarian but also the subject owner to which the disclosure is not desired may view the relative health information of the pet of the relative owner during the display of the electronic medical record.

In such a case, in the medical care support device 10 according to the present embodiment, only the information representing the presence or absence of relative health information of the relative (relative health information absence information 68 or relative health information presence information 70) is displayed in the electronic medical record information 23B displayed on the display unit 24. Therefore, it is possible to display the relative health information of the relative by the display instruction of the user and to avoid the disclosure of the information to subject owner since the relative health information of the relative which is the personal information is not carelessly displayed on the display unit 24.

Therefore, with the medical care support device 10 according to the present embodiment, it is possible to efficiently perform appropriate medical care support for the subject while personal information is protected.

With the medical care support device 10 according to the present embodiment, the user can perform the medical care based on the relative health information of the relative of the subject. Therefore, it is possible to perform more effective medical care as compared with a case where the medical care is performed simply according to the dog breed (breed). The user can perform not only the medical treatment of the disease suffered by the subject but also measures to prevent a disease more likely to be suffered. Therefore, it is possible to prolong the life of the subject and further to suppress medical costs.

In the above embodiment, the administrator or the like registers the relative health information that can be browsed in the authentication information 37 in advance by associating the user who is permitted to browse with the user ID to restrict the disclosure of the relative health information. However, a method of restricting the disclosure of the relative health information is not limited to this example. In a case where the relative health information of the subject is registered, a form may be employed in which the owner of the subject can limit the relative health information permitted to be browsed.

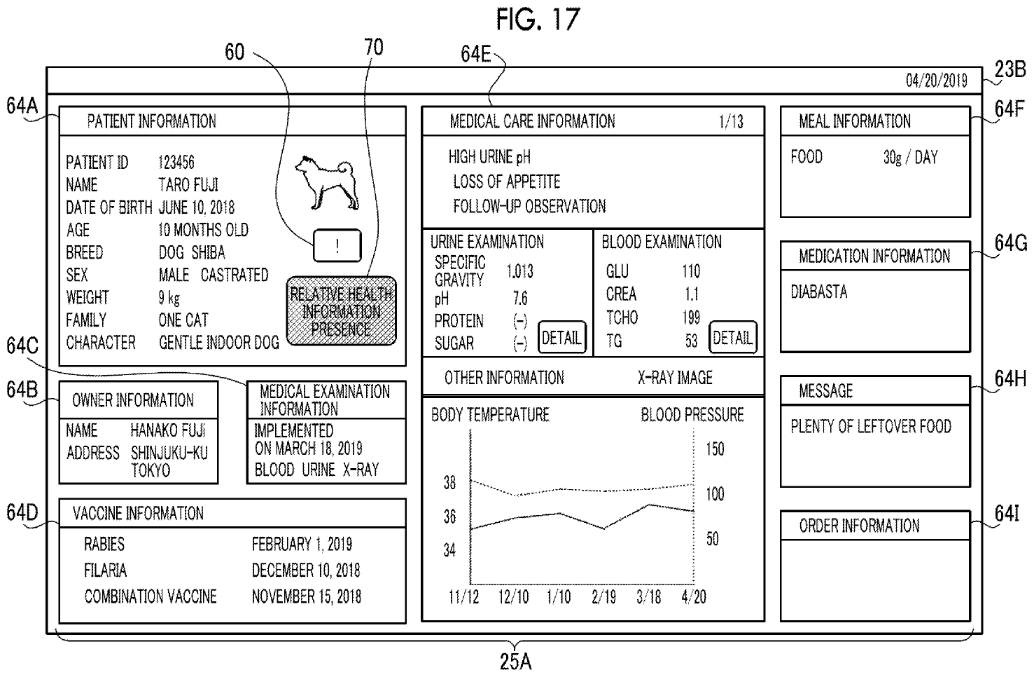

In the present embodiment, the form has been described in which the presence or absence of the relative health information is displayed on the display unit 24 as the electronic medical record information 23B. Further, a form may be employed in which information notifying an update is displayed on the display unit 24 in a case where the relative health information of the relative is updated. For example, as an example shown in FIG. 17, a form may be employed in which a color of the display area of the relative health information presence information 70 displayed on the display unit 24 is changed to a color different from normal or the display area thereof blinks to notify that the relative health information of the relative is updated. Alternatively, for example, a form may be employed in which a mark representing that the relative health information of the relative is updated is displayed near the display area of the relative health information presence information 70 or the like. An example of the update of relative health information of the relative includes a case where the relative health information of the relative is newly registered in the medical care support system 1 and a case where the relative suffers from a disease and receives the medical treatment and thus the health information 34 is updated.

The following various processors may be used as a hardware structure of a processing unit that executes various types of processing such as each functional unit of the medical care support device 10 in the above embodiments. The various processors include a programmable logic device (PLD) which is a processor whose circuit configuration is changeable after manufacturing such as a field-programmable gate array (FPGA), a dedicated electric circuit which is a processor having a circuit configuration exclusively designed to execute specific processing such as an application specific integrated circuit (ASIC), and the like, in addition to the CPU which is a general-purpose processor that executes software (program) to function as various processing units as described above.

One processing unit may be configured by one of the various processors or a combination of two or more processors of the same type or different types (for example, a combination of a plurality of FPGAs or a combination of a CPU and an FPGA). A plurality of processing units may be configured by one processor.

As an example of configuring the plurality of processing units with one processor, first, there is a form in which one processor is configured by a combination of one or more CPUs and software and the processor functions as the plurality of processing units, as represented by computers such as a client and a server. Second, there is a form in which a processor that realizes the functions of the entire system including the plurality of processing units with one integrated circuit (IC) chip is used, as represented by a system-on-chip (SoC) or the like. As described above, the various processing units are configured using one or more of the various processors as the hardware structure.

Further, more specifically, a circuitry combining circuit elements such as semiconductor elements can be used as the hardware structure of the various processors.

In the above embodiments, the form has been described in which the medical care support program 23A and the proposal program 23C are stored (installed) in the storage unit 22 in advance. However, the present disclosure is not limited thereto. The medical care support program 23A and the proposal program 23C may be provided in a form recorded on a recording medium such as a compact disc read only memory (CD-ROM), a digital versatile disc read only memory (DVD-ROM), or a Universal Serial Bus (USB) memory. The medical care support program 23A and the proposal program 23C may be downloaded from an external device through a network.

What is claimed is:

1. A medical care support device comprising:
a first display control unit that performs control of displaying, on a display unit, electronic medical record information including first information used for medical care of a subject and second information representing presence or absence of relative health information on health of a relative of the subject;
a reception unit that receives a display instruction performed according to the display of the second information to display the relative health information on the display unit; and
a second display control unit that performs control for displaying the relative health information on the display unit in a case where the reception unit receives the display instruction.

2. The medical care support device according to claim 1,
wherein the second display control unit displays the relative health information in a case where the reception unit further receives identification information for identifying a person permitted to browse the relative health information.

3. The medical care support device according to claim 2,
wherein the relative health information includes, for each relative of the subject, at least two or more of relative relationship information representing a relationship with the subject, medical history information, medical treatment information, health diagnosis information, or gene information, and
wherein the identification information is in correspondence with information that is permitted to be browsed among a plurality of pieces of information included in the relative health information.

4. The medical care support device according to claim 1,
wherein the relative health information includes, for each relative, at least one of information representing a relationship with the subject, medical history information, medical treatment information, health diagnosis information, or gene information.

5. The medical care support device according to claim 1, further comprising:
a proposal unit that proposes at least one of an examination related to a disease of the subject which is a medical care target or a medical care policy based on the relative health information.

6. The medical care support device according to claim 1,
wherein the subject is an animal other than a human and a breed of the animal for which the browsing of the relative health information is recommended is determined in advance, and
wherein the first display control unit performs control of further displaying, on the display unit, browsing recommended information representing that the browsing is recommended in a case where the breed of the subject is a breed for which the browsing of the relative health information is recommended.

7. The medical care support device according to claim 6,
wherein the breed of the animal for which the browsing of the relative health information is recommended is a breed within a predetermined higher order in ranking of at least one of a breeding number or a sales number.

8. The medical care support device according to claim 1, further comprising:
an acquisition unit that acquires the relative health information of the subject based on information for identifying a relative in correspondence with a subject which is a medical care target, from correspondence relationship information stored in an information storage unit that stores a correspondence relationship between a plurality of subjects and relative health information of each of the plurality of subjects.

9. The medical care support device according to claim 8,
wherein the information for identifying the relative is information included in a pedigree paper of the subject which is the medical care target.

10. The medical care support device according to claim 8,
wherein the first display control unit performs control of further displaying, on the display unit, recommended storage information recommending that the correspondence relationship is stored in the information storage unit in a case where the correspondence relationship is not stored in the information storage unit for the subject which is the medical care target.

11. A medical care support method executed by a computer, comprising:
displaying, on a display unit, electronic medical record information including first information used for medical care of a subject and second information representing presence or absence of relative health information on health of a relative of the subject;
receiving a display instruction performed according to the display of the second information to display the relative health information on the display unit; and
displaying the relative health information on the display unit in a case where the reception unit receives the display instruction.

12. A non-transitory computer-readable storage medium storing a medical care support program causing a computer to execute the following processing of:
displaying, on a display unit, electronic medical record information including first information used for medical care of a subject and second information representing presence or absence of relative health information on health of a relative of the subject;
receiving a display instruction performed according to the display of the second information to display the relative health information on the display unit; and
displaying the relative health information on the display unit in a case where the reception unit receives the display instruction.
